(19) United States
(12) Patent Application Publication
(10) Pub. No.: US 2005/0153317 A1
(43) Pub. Date: Jul. 14, 2005

(54) Methods and systems for inferring traits to breed and manage non-beef livestock

(75) Inventors: Sue DeNise, Davis, CA (US); David Rosenfeld, Sacramento, CA (US); Richard Kerr, Davis, CA (US); Stephen Bates, Davis, CA (US); Tom Holm, Salt Lake City, UT (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: Metamorphix, Inc., Savage, MD (US)

(21) Appl. No.: 10/972,079

(22) Filed: Oct. 22, 2004

Related U.S. Application Data
(60) Provisional application No. 60/514,333, filed on Oct. 24, 2003.

(51) Int. Cl. C12Q 1/68

(52) U.S. Cl. 435/6

(57) ABSTRACT

Methods and systems are provided for managing non-beef livestock subjects in order to maximize their individual potential performance and the value of a product from the non-beef livestock subjects, and to maximize profits obtained in marketing the non-beef livestock subjects. The methods and systems draw an inference of a trait of a non-beef livestock subject by determining the nucleotide occurrence of at least one non-beef livestock SNP that is determined to be associated with the trait. The inference is used in methods of the present invention to establish the economic value of a non-beef livestock subject, to improve profits related to selling beef from a non-beef livestock subject; to manage non-beef livestock subjects, to sort non-beef livestock subjects; to improve the genetics of a non-beef livestock population by selecting and breeding of non-beef livestock subjects, to clone a non-beef livestock subject with a specific trait, to track meat or another commercial product of a non-beef livestock subject; and to diagnose a health condition of a non-beef livestock subject. Certain embodiments of the present invention provide methods, systems, and kits are directed to inferences of a trait related to milk or a dairy product in a livestock subject.

### CROSS REFERENCE TO RELATED APPLICATIONS

This application claims the benefit of priority under 35 U.S.C. § 119(e) of U.S. Ser. No. 60/514,333, filed Oct. 24, 2003, the entire content of which is incorporated herein by reference.

### BACKGROUND OF THE INVENTION

1. Field of the Invention

The invention relates generally to genomic association analyses and more specifically to the use of single nucleotide polymorphisms as a determinant of trait identification for management, selection and mating system of non-beef livestock.

2. Background Information

Currently there are no cost effective methods for identifying non-beef livestock that give accurate prediction of the genetic potential to produce products such as meat or dairy products. Such information could be used for example for chicken or swine breeders to identify desirable animals for breeding. The information could also be used by chicken or swine processors to select or value animals. Thus, it is desirable to have a method that can be used to assess the potential of live non-beef livestock, particularly young non-beef livestock well in advance of the arrival of the animal at the packing house.

Therefore, there remains a need for cost effective methods for identifying non-beef livestock that are based on genetic information to draw accurate inferences regarding traits the non-beef livestock.

### SUMMARY OF THE INVENTION

In order to solve the previous problems, the present invention provides methods and systems for managing, selecting and mating non-beef livestock. These methods for identification and monitoring of key characteristics of individual animals and management of individual animals maximize their individual potential performance and product value. The methods of invention provide systems to collect, record and store such data by individual animal identification so that it is usable to improve future animals bred by the breeder. The methods and systems of the present invention utilize information regarding genetic diversity among non-beef livestock, particularly single nucleotide polymorphisms (SNPs), and the effect of nucleotide occurrences of SNPs on important traits.

The present invention further provides methods for selecting a given animal for shipment at the optimum time, considering the animal's condition, performance and market factors, the ability to grow the animal to its optimum individual potential of physical and economic performance, and the ability to record and preserve each animal's performance history in during processing for use in cultivating and managing current and future animals for production of various products including meat and dairy products. These methods allow management of the current diversity of chickens and swine, for example, to improve the chicken and pork product quality and uniformity, thus improving revenue generated from sales of these products.

This invention identifies animals that have superior traits, predicted very accurately, that can be used to identify parents of the next generation through selection. These methods for example, can be used to create pure lines of chickens or pigs which could be used to produce meat chickens or pigs, respectively. Therefore, the improved traits would, through time, flow to the entire population of animals. This invention provides a method for determining the optimum male and female parent to maximize the genetic components of dominance and epistasis thus maximizing heterosis and hybrid vigor in the market animals.

The present invention in certain embodiments provides a method for inferring a trait of a non-beef livestock subject from a nucleic acid sample of the subject. The method includes identifying in the nucleic acid sample, at least one nucleotide occurrence of a single nucleotide polymorphism (SNP). The nucleotide occurrence is associated with the trait, thereby inferring the trait. The nucleotide occurrence of at least 2 SNPs can be determined.

The SNPs can make up a haplotype and the method can identify a haplotype allele that is associated with the trait. Furthermore, the method can include identifying a diploid pair of haplotype alleles.

The non-beef livestock subject can be an alpaca, a buffalo, a cow, a goat, a horse, a llama, a sheep, a pig, an ostrich, a chicken, a turkey, an elk, an emu, a deer, a lamb, or a duck. In certain embodiments, the non-beef livestock subject is a pig. In these embodiments, the trait can be age at puberty, reproductive potential, number of pigs farrowed alive, birth weight of pigs farrowed, longevity, weight of subject at a target timepoint, number of pigs weaned, percent of pigs weaned, pigs marketed/sow/year, average weaning weight of pigs, rate of gain, days to a target weight, meat quality, fiber quality, fiber yield, feed efficiency, manure characteristic, muscle content, fat content (leanness), disease resistance, disease susceptibility, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, optimal diet, or conception rate. Manure characteristics include quantity, organic matter, plant nutrients, or salts.

In certain embodiments, the non-beef livestock subject is a bird or avian species. For example, the bird or avian species can be a chicken or a turkey. In these embodiments, the trait can be egg production, feed efficiency, livability, meat yield, longevity, white meat yield, dark meat yield, disease resistance, disease susceptibility, optimal diet time to maturity, time to a target weight, weight at a target timepoint, average daily weight gain, meat quality, muscle content, fat content, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, or conception rate. In one embodiment, the trait is resistance to Salmonella infection, ascites, and listeria infection.

In certain embodiments, the non-beef livestock subject is a bird or avian species that produces eggs for mammalian consumption. In certain embodiments, the bird or avian species is a chicken and the trait can be a characteristic of an egg of the bird or a characteristic of a product of the egg.

The egg characteristic can be quality, size, shape, shelf-life, freshness, cholesterol content, color, biotin content, calcium content, shell quality, yolk color, lecithin content, number of yolks, yolk content, white content, vitamin content, vitamin D content, nutrient density, protein content, albumen content, protein quality, avidin content, fat content, saturated fat content, unsaturated fat content, interior egg quality, number of blood spots, air cell size, grade, a bloom characteristic, chalaza prevalence or appearance, ease of peeling, likelihood of being a restricted egg, Salmonella content.

The inferences discussed above, can be used for the following aspects of the invention: to establish the economic value of a non-beef livestock subject; to improve profits related to selling a product from a non-beef livestock subject; to manage non-beef livestock subjects; to sort non-beef livestock subjects; to improve the genetics of a non-beef livestock population by selecting and breeding of non-beef livestock subjects; to clone a non-beef livestock subject with a specific trait, a combination of traits, or a combination of SNP markers that predict a trait; to track meat or another commercial product of a non-beef livestock subject; to certify a specific product based on known characteristics; to diagnose a health condition of a non-beef livestock subject; and to select a pig or other non-beef species for use in xenotransplantation.

In another aspect, the present invention provides a method for identifying a non-beef livestock genetic marker that influences a trait. The method includes analyzing non-beef livestock genetic markers for association with the trait. The method can also involve determining nucleotide occurrences of at least two SNPs that influence the trait or a group of traits.

In another aspect, the present invention provides a high-throughput system for determining the nucleotide occurrences at a series of non-beef livestock single nucleotide polymorphisms (SNPs). The system includes one of the following: solid support to which a series of oligonucleotides can be directly or indirectly attached, homogeneous assay medium and a microfluidic device. The system is used to determine the nucleotide occurrence of non-beef livestock SNPs that are associated with a trait.

In another aspect, the present invention provides a computer system that includes a database having records containing information regarding a series of non-beef livestock single nucleotide polymorphisms (SNPs), and a user interface allowing a user to input nucleotide occurrences of the series of SNPs for a non-beef livestock subject. The user interface can be used to query the database and display results of the query. The database can include records representing some or all of the SNPs of a non-beef livestock SNP map, which can be a high-density non-beef SNP map. The database can also include information regarding haplotypes and haplotype alleles from the SNPs. Furthermore, the database can include information regarding traits and/or traits that are associated with some or all of the SNPs and/or haplotypes. In these embodiments the computer system can be used, for example, for any of the aspects of the invention that infer a trait of a non-beef livestock subject.

Certain embodiments of the present invention provide methods, systems, and kits identical to those discussed above, and herein, except that the trait is milk production, a trait affecting milk production, a characteristic of milk, a characteristic of a dairy product, milk component composition, or mastitis resistance. In these embodiments, the methods, systems, and kits relate to all livestock (i.e. they include beef subjects).

Accordingly, in certain embodiments, the present invention provides a method for inferring from a nucleic acid sample of a livestock, a trait of milk production, a trait affecting milk production, a characteristic of milk, a characteristic of a dairy product, milk component composition including fat, protein, and bioreactive molecules, or mastitis resistance, for the livestock. The method includes identifying in the nucleic acid sample, at least one nucleotide occurrence of a single nucleotide polymorphism (SNP), wherein the nucleotide occurrence is associated with the trait and wherein the trait is thereby inferred.

The livestock subject can be, for example, a cow, a goat, a sheep, a buffalo, a camel, a horse, or a deer. The trait can be, for example, milk protein content, milk fat content, milk amino acid profile, milk fatty acid profile, bioreactive molecule content, milk taste appeal, or taste appeal of a dairy product. Furthermore, the trait can be taste appeal of milk, cheese, yogurt, cream, butter, or ice cream. Alternatively, the trait can be milk or dairy product solids content, calcium content, riboflavin content, nitrogen potassium content, protein content, casein content, fat content, whey content, vitamin A content, vitamin D content, or phosphorus content. The trait can also be lactation period or production in milk of a transgenic protein or transgenically-produced pharmaceutical product.

In one aspect, the methods of the invention can be utilized in combination with various hypermutable sequences, such as microsatellite nucleic acid sequences to infer traits of non-beef livestock. As used herein, the term “hypermutable” refers to a nucleic acid sequence that is susceptible to instability, thus resulting in nucleic acid alterations. Such alterations include the deletion and addition of nucleotides. The hypermutable sequences of the invention are most often microsatellite DNA sequences which, by definition, are small tandem repeat DNA sequences. Thus, a combination of SNP analysis and microsatellite analysis may be used to infer a trait(s) of a non-beef livestock subject.

In another embodiment, a method for identifying the parentage of a non-beef test subject is provided. The method includes obtaining a nucleic acid sample from the test subject and identifying in the nucleic acid sample at least one single nucleotide polymorphism (SNP) corresponding to the nucleotide at position 600 of any one of SEQ ID NOs:1-96,631, or the complement thereof. The method optionally includes repeating the identification for additional subjects. The method further includes determining the alleles corresponding to each SNP identified and comparing the alleles to putative parents of the test subject. Generally parents not possessing at least one allele in common with the test subject are excluded. The non-beef livestock subject can be derived from an avian species, including chickens or turkeys.

In another embodiment, a method for determining the identity of a non-beef test subject is provided. The method includes obtaining a nucleic acid sample from the test subject by a method comprising identifying in the nucleic acid sample at least one single nucleotide polymorphism (SNP) corresponding to the nucleotide at position 600 of any one of SEQ ID NOs:1-96,631, or the complement thereof. The method optionally includes repeating the identification for additional subjects. The method further includes determining the two alleles corresponding to each SNP identified and comparing the alleles to the alleles identified in a known sample previously obtained from the test subject.

In another embodiment a method to infer breed or line of a non-beef test subject from a nucleic acid sample obtained from the subject is provided. The method includes identifying in the nucleic acid sample, at least one nucleotide occurrence of at least one single nucleotide polymorphism (SNP) corresponding to the nucleotide at position 600 of any one of SEQ ID NOS:1-96,631.

In another embodiment, a method of generating a genome discovery map is provided. The method includes selecting a plurality of single nucleotide polymorphism (SNP) markers selected from at least two of the SNP markers at position 600 of any of SEQ ID NOs:1-96,631. Generally, each marker in the series will be separated by approximately 150,000 bp. The method further includes generating the genome discovery map based upon the selected markers. In an exemplary aspect, the genome discovery map is a whole genome discovery map. The plurality of single nucleotide polymorphism (SNP) markers can includes about 10, 100, 1000, 8000 or 10000 markers. The plurality of single nucleotide polymorphism (SNP) markers, or the number of markers indicated by the amount of linkage disequilibrium in each non-beef species, are can further be selected based upon dispersion across the entire genome.

In another embodiment, a kit for determining nucleotide occurrences of non-beef SNPs is provided. In general, the kit can contain an oligonucleotide probe, primer, or primer pair, or combinations thereof, for identifying the nucleotide occurrence of at least one non-beef single nucleotide polymorphism (SNP) corresponding to position 600 of any one SE Q ID NOs:1-96,631, or complement thereof. The kit can further include one or more detectable labels.

In another embodiment, a database comprising a plurality of single nucleotide polymorphisms (SNP) selected from at least two of the SNP markers at position 600 of any of SEQ ID NOs:1-96,631, or complement thereof, is provided. Also provided is a database that includes allele frequencies generated by analyzing the SNP database.

In another embodiment, an isolated single nucleotide polymorphism (SNP) corresponding to a nucleotide at position 600 of any one of SEQ ID NOs:1-96,631, or the complement thereof, is provided. Also provided is an isolated oligonucleotide comprising a nucleotide corresponding to a nucleotide at position 600 of any one of SEQ ID NOs:1-96,631, or the complement thereof. Also provided is an isolated oligonucleotide comprising any one of SEQ ID NOs:1-96,631 and an isolated oligonucleotide selected from the group consisting of SEQ ID NOs:1-96,631. The invention further encompasses the complement of the aforementioned oligonucleotides.

In another embodiment, a panel comprising at least one single nucleotide polymorphism (SNP) corresponding to a nucleotide at position 600 of any one of SEQ ID NOs:1-96,631, or the complement thereof, is provided.

In yet another embodiment, a computer-based method for identifying or inferring a trait of a non-beef test subject is provided. The method includes obtaining a nucleic acid sample from the non-beef subject and identifying in the nucleic acid sample at least one nucleotide occurrence of at least one single nucleotide polymorphism (SNP) corresponding to position 600 of any one of SEQ ID NOs:1-96,631, or complement thereof. The method further includes searching a database that includes a plurality of single nucleotide polymorphism (SNP) markers selected from at least two of the SNP markers at position 600 of any of SEQ ID NOs:1-96,631, wherein the database is generated from a nucleic acid sample obtained from a non-beef non-test subject. The method further includes retrieving the information from the database and optionally storing the information in a memory location associated with a user such that the information may be subsequently accessed and viewed by the user.

### DETAILED DESCRIPTION OF THE INVENTION

The specification hereby incorporates by reference in their entirety, the files contained on the two compact discs filed herewith. The first compact disc includes a file entitled “MMI1110-2 Chicken SNP Table 1.txt,” created Oct. 12, 2004, which is 6,736 kilobytes in size. The second disc includes a sequence listing which is included in a file entitled “MMI110-2 Sequence Listing.txt,” created Oct. 12, 2004, which is 79,891 kilobytes in size. Duplicates of the aforementioned discs contain the appropriately labeled file.

The methods of the invention are particularly well suited for managing, selecting or mating non-beef livestock subjects. The methods allow for the ability to identify and monitor key characteristics of individual animals and manage those individual animals to maximize their individual potential performance or the value of products derived from the animals. Furthermore, the methods of the inventions provide systems to collect, record and store such data by individual animal identification so that it is usable to improve future animals bred by a breeder and processed by a processor. Specific embodiments of the invention are exemplified in Exhibit A and Exhibit B, as provided in U.S. Ser. No. 60/514,333, filed Oct. 24, 2003, and incorporated herein by reference.

The methods and systems allow for the ability to identify and monitor key characteristics of individual animals and manage those individual animals to maximize their individual potential performance and product value. Furthermore, the methods of the inventions provide systems to collect, record and store such data by individual animal identification so that it is usable to improve future animals bred by the producer and managed by the feedlot. These methods can utilize computer models to utilize information regarding nucleotide occurrences of SNPs and their association with traits, to predict an economic value for a non-beef livestock subject.

Accordingly, a method according to this aspect of the invention includes inferring a trait of the non-beef livestock subject from a nucleic acid sample of the non-beef livestock subject. The inference is drawn by a method that includes identifying in the sample, a nucleotide occurrence for at least one single nucleotide polymorphism (SNP), wherein the nucleotide occurrence is associated with the trait; and wherein the trait affects the physical characteristic. Furthermore, the method includes managing at least one of food intake, diet composition, administration of feed additives or pharmacological treatments such as vaccines, antibiotics, hormones and other metabolic modifiers, age and weight at which diet changes or pharmacological treatments are imposed, days fed specific diets, castration, feeding methods and management, imposition of internal or external measurements and environment of the non-beef livestock subject based on the inferred trait. This management results in a maximization of physical characteristic of a non-beef livestock subject, for example to obtain a maximum amount of high grade pork from a pig, and/or to increase the chances of obtaining high grade pork with excellent tenderness and high yield from the pig, taking into account the inputs required to reach those endpoints.

The method can be used to discriminate among those animals where growth implants, vitamins, and other interventions could provide the greatest value. For example, animals that do not have the traits to reach high quality pork may be given growth implants until the end of a feeding period, thus maximizing feed efficiency.

The method also allows a processor to predict the quality and yield grades of non-beef livestock in the system to optimize marketing of the fed animal or the product to meet target market specification. The method also provides information to the processor for purchase decisions based on the predicted economic returns from a specific supplier. Furthermore, The method allows the creation of integrated programs spanning breeders, processors, packers, and retailers.

The present invention further provides methods for selecting a given animal for shipment at the optimum time, considering the animal's condition, performance and market factors, the ability to grow the animal to its optimum individual potential of physical and economic performance, and the ability to record and preserve each animal's performance history in the feedlot and carcass data from the packing plant for use in cultivating and managing current and future animals for production of various products such as pork and eggs. These methods allow management of the current diversity of non-beef livestock to improve the quality and uniformity of products from the non-beef livestock, thus improving revenue generated from sales of the products.

The methods can use a bioeconomic valuation method that establishes the economic value of a non-beef livestock subject, or a group of non-beef livestock subjects, to optimize profits from production of products from the subjects. Accordingly, in another aspect, the present invention provides a method for establishing the economic value of a non-beef livestock subject. According to the method, an inference is drawn regarding a trait of the non-beef livestock subject from a nucleic acid sample of the non-beef livestock subject. The inference is drawn by a method that includes identifying nucleotide occurrences for at least one single nucleotide polymorphism (SNP), wherein the nucleotide occurrence is associated with the trait, and wherein the trait affects the value of the non-beef livestock subject.

The method includes identification of the causative mutation influencing the trait directly or the determination of 1 or more SNPs that are in linkage disequilibrium with the associated trait.

The method can include a determination of the nucleotide occurrence of at least 2 SNPs. At least 2 SNPs can form all or a portion of a haplotype, wherein the method identifies a haplotype allele that is in linkage disequilibrium and thus associated with the trait. Furthermore, the method can include identifying a diploid pair of haplotype alleles.

A method according to this aspect of the invention can further include using traditional factors affecting the economic value of the non-beef livestock subject in combination with the inference based on nucleotide occurrence data to determine the economic value of the non-beef livestock subject.

As used herein, the term “at least one”, when used in reference to a gene, SNP, haplotype, or the like, means 1, 2, 3, 4, 5, 6, 7, 8, 9, 10, etc., up to and including all of the haplotype alleles, genes, and/or SNPs of the non-beef livestock genome. Reference to “at least a second” gene, SNP, or the like, means two or more, i.e., 2, 3, 4, 5, 6, 7, 8, 9, 10, etc., non-beef livestock genes, SNPs, or the like.

Polymorphisms are allelic variants that occur in a population that can be a single nucleotide difference present at a locus, or can be an insertion or deletion of one, a few or many consecutive nucleotides. As such, a single nucleotide polymorphism (SNP) is characterized by the presence in a population of one or two, three or four nucleotides (i.e., adenosine, cytosine, guanosine or thymidine), typically less than all four nucleotides, at a particular locus in a genome such as a non-beef livestock genome. It will be recognized that, while the methods of the invention are exemplified primarily by the detection of SNPs, the disclosed methods or others known in the art similarly can be used to identify other types of non-beef livestock polymorphisms, which typically involve more than one nucleotide.

The term “haplotypes” as used herein refers to groupings of two or more SNPs that are physically present on the same chromosome which tend to be inherited together except when recombination occurs. The haplotype provides information regarding an allele of the gene, regulatory regions or other genetic sequences affecting a trait The linkage disequilibrium and, thus, association of a SNP or a haplotype allele(s) and a non-beef livestock trait can be strong enough to be detected using simple genetic approaches, or can require more sophisticated statistical approaches to be identified.

Numerous methods for identifying haplotype alleles in nucleic acid samples are known in the art. In general, nucleic acid occurrences for the individual SNPs are determined, and then combined to identify haplotype alleles. The Stephens and Donnelly algorithm (Am. J. Hum. Genet. 68: 978-989, 2001, which is incorporated herein by reference) can be applied to the data generated regarding individual nucleotide occurrences in SNP markers of the subject, in order to determine alleles for each haplotype in a subject's genotype. Other methods can be used to determine alleles for each haplotype in the subject's genotype, for example Clarks algorithm, and an EM algorithm described by Raymond and Rousset (Raymond et al. 1994. GenePop. Ver 3.0. Institut des Siences de l'Evolution. Universite de Montpellier, France. 1994).

As used herein, the term “infer” or “inferring”, when used in reference to a trait, means drawing a conclusion about a trait using a process of analyzing individually or in combination, nucleotide occurrence(s) of one or more SNP(s), which can be part of one or more haplotypes, in a nucleic acid sample of the subject, and comparing the individual or combination of nucleotide occurrence(s) of the SNP(s) to known relationships of nucleotide occurrence(s) of the SNP(s) and the trait. As disclosed herein, the nucleotide occurrence(s) can be identified directly by examining nucleic acid molecules, or indirectly by examining a polypeptide encoded by a particular genomic where the polymorphism is associated with an amino acid change in the encoded polypeptide.

Relationships between nucleotide occurrences of one or more SNPs or haplotypes and a trait can be identified using known statistical methods. A statistical analysis result which shows an association of one or more SNPs or haplotypes with a trait with at least 80%, 85%, 90%, 95%, or 99%, or 95% confidence, or alternatively a probability of insignificance less than 0.05, can be used to identify SNPs and haplotypes. These statistical tools may test for significance related to a null hypothesis that an on-test SNP allele or haplotype allele is not significantly different between groups with different traits. If the significance of this difference is low, it suggests the allele is not related to a trait.

As another example, associations between nucleotide occurrences of one or more SNPs or haplotypes and a trait (i.e. selection of significant markers) can be identified using a two part analysis. In the first part, DNA from animals at the extremes of a trait are pooled, and the allele frequency of one or more SNPs or haplotypes for each tail of the distribution is estimated. Alleles of SNPs and/or haplotypes that are apparently associated with extremes of a trait are identified and are used to construct a candidate SNP and/or haplotype set. Statistical cut-offs are set relatively low to assure that significant SNPs and/or haplotypes are not overlooked during the first part of the method.

During the second stage, individual animals are genotyped for the candidate SNP and/or haplotype set. The second stage is set up to account for as much of the genetic variation as possible in a specific trait without introducing substantial error. This is a balancing act of the prediction process. Some animals are predicted with high accuracy and others with low accuracy.

In diploid organisms such as non-beef livestock, somatic cells, which are diploid, include two alleles for each single-locus haplotype. As such, in some cases, the two alleles of a haplotype are referred to herein as a genotype or as a diploid pair, and the analysis of somatic cells, typically identifies the alleles for each copy of the haplotype. Methods of the present invention can include identifying a diploid pair of haplotype alleles. These alleles can be identical (homozygous) or can be different (heterozygous). Haplotypes that extend over multiple loci on the same chromosome include up to 2 the Nth power alleles where N is the number of loci. It is beneficial to express polymorphisms in terms of multi-locus (i.e. multi SNP) haplotypes because haplotypes offer enhanced statistical power for genetic association studies. Multi-locus haplotypes can be precisely determined from diploid pairs when the diploid pairs include 0 or 1 heterozygous pairs, and N or N−1 homozygous pairs. When multi-locus haplotypes cannot be precisely determined, they can sometimes be inferred by statistical methods. Methods of the invention can include identifying multi-locus haplotypes, either precisely determined, or inferred.

A sample useful for practicing a method of the invention can be any biological sample of a subject, typically a non-beef livestock subject, that contains nucleic acid molecules, including portions of the genomic sequences to be examined, or corresponding encoded polypeptides, depending on the particular method. As such, the sample can be a cell, tissue or organ sample, or can be a sample of a biological material such as blood, milk, semen, saliva, hair, tissue, and the like. A nucleic acid sample useful for practicing a method of the invention can be deoxyribonucleic (DNA) acid or ribonucleic acids (RNA). The nucleic acid sample generally is a deoxyribonucleic acid sample, particularly genomic DNA or an amplification product thereof. However, where heteronuclear ribonucleic acid which includes unspliced mRNA precursor RNA molecules and non-coding regulatory molecules such as RNA is available, a cDNA or amplification product thereof can be used.

Where each of the SNPs of the haplotype is present in a coding region of a gene(s), the nucleic acid sample can be DNA or RNA, or products derived therefrom, for example, amplification products. Furthermore, while the methods of the invention generally are exemplified with respect to a nucleic acid sample, it will be recognized that particular haplotype alleles can be in coding regions of a genomic and can result in polypeptides containing different amino acids at the positions corresponding to the SNPs due to non-degenerate codon changes. As such, in another aspect, the methods of the invention can be practiced using a sample containing polypeptides of the subject.

In one embodiment, DNA samples are collected and stored in a retrievable barcode system, either automated or manual, that ties to a database. Collection practices include systems for collecting tissue, hair, mouth cells or blood samples from individual animals at the same time that ear tags, electronic identification or other devices are attached or implanted into the animal. Tissue collection devices can be integrated into the tool used for placing the ear tag. Body fluid samples are collected and can be stored on a membrane bound system. All methods could be automatically uploaded into a primary database.

The sample is then analyzed on the premises or sent to a laboratory where a high-throughput genotyping system is used to analyze the sample. Traits are predicted in the field in real-time or in the laboratory and forwarded to the field. Processors then uses this information to sort and manage animals to maximize profitability and marketing potential.

The present invention can also be used to provide information to breeders to make breeding, mating, and or cloning decisions. This invention can also be combined with traditional genetic evaluation methods to improve selection, mating, or cloning strategies.

The subject of the present invention can be any non-beef livestock subject. The non-beef livestock subject can be, for example, an alpacas, a buffalo, a cow, a goat, a horse, a llama, a sheep, a pig, an ostrich, a chicken, a turkey, an elk, an emu, a deer, a lamb, or a duck. As discussed below, in embodiments where the trait is related to milk or a dairy product, the subject can be any livestock subject including a beef subject.

For methods of the invention directed at sorting non-beef livestock subjects, managing non-beef livestock subjects, improving profits related to selling meat from a non-beef livestock subject, the animal can be a young non-beef livestock subject ranging in ages from conception to the time the animal is harvested and meat and other commercial products obtained.

A “trait” is a characteristic of an organism that manifests itself in a phenotype. Many traits are the result of the expression of a single gene, but some are polygenic (i.e., result from simultaneous expression of more than one gene). A “phenotype” is an outward appearance or other visible characteristic of an organism. Many different non-beef livestock traits can be inferred by methods of the present invention.

In certain embodiments, the non-beef livestock subject is a pig. In these embodiments, the trait can be age at puberty, reproductive potential, number of pigs farrowed alive, birth weight of pigs farrowed, longevity, weight of subject at a target timepoint, number of pigs weaned, percent of pigs weaned, pigs marketed/sow/year, average weaning weight of pigs, rate of gain, days to a target weight, meat quality, feed efficiency, manure characteristic, muscle content, fat content (leanness), disease resistance, disease susceptibility, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, optimal diet, or conception rate. Manure characteristics include quantity, organic matter, plant nutrients, or salts.

In certain embodiments, the non-beef livestock subject is a bird or avian species. For example, the bird or avian species can be a chicken or a turkey. In these embodiments, the trait can be egg production, feed efficiency, livability, meat yield, longevity, white meat yield, dark meat yield, disease resistance, disease susceptibility, optimal diet time to maturity, time to a target weight, weight at a target timepoint, average daily weight gain, meat quality, muscle content, fat content, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, or conception rate. In one embodiment, the trait is resistance to Salmonella infection, ascites, and listeria infection.

In certain embodiments, the non-beef livestock subject is a bird or avian species that produces eggs for mammalian consumption. In certain embodiments, the bird or avian species is a chicken and the trait can be a characteristic of an egg of the bird or a characteristic of a product of the egg.

The egg characteristic can be quality, size, shape, shelf-life, freshness, cholesterol content, color, biotin content, calcium content, shell quality, yolk color, lecithin content, number of yolks, yolk content, white content, vitamin content, vitamin D content, nutrient density, protein content, albumen content, protein quality, avidin content, fat content, saturated fat content, unsaturated fat content, interior egg quality, number of blood spots, air cell size, grade, a bloom characteristic, chalaza prevalence or appearance, ease of peeling, likelihood of being a restricted egg, Salmonella content.

Methods of the present invention can be used to infer more than one trait. For example a method of the present invention can be used to infer a series of traits. As used herein, a phenotype and a trait may be used interchangeably in some instances. Accordingly, a method of the present invention can infer, for example, quality grade, muscle content, and feed efficiency. This inference can be made using one SNP or a series of SNPs. Thus, a single SNP can be used to infer multiple traits; multiple SNPs can be used to infer multiple traits; or a single SNP can be used to infer a single trait.

In another aspect, the present invention provides a method for improving profits related to selling meat from a non-beef livestock subject. The method includes drawing an inference regarding a trait of the non-beef livestock subject from a nucleic acid sample of the non-beef livestock subject. The method is typically performed by a method that includes identifying a nucleotide occurrence for at least one single nucleotide polymorphism (SNP), wherein the nucleotide occurrence is associated with the trait, and wherein the trait affects the value of the animal or its products. Furthermore, the method includes managing at least one of food intake, diet composition, administration of feed additives or pharmacological treatments such as vaccines, antibiotics, hormones and other metabolic modifiers, age and weight at which diet changes or pharmacological treatments are imposed, days fed specific diets, castration, feeding methods and management, imposition of internal or external measurements and environment of the non-beef livestock subject based on the inferred trait. Then at least one non-beef livestock commercial product, typically meat or milk, is obtained from the non-beef livestock subject.

Methods according to this aspect of the present invention can utilize a bioeconomic model, such as a model that estimates the net value of one or more non-beef livestock subjects based on one or more traits. By this method, traits of one, or a series of traits are inferred, for example, an inference regarding several characteristics of meat that will be obtained from the non-beef livestock subject. The inferred trait information then can be entered into a model that uses the information to estimate a value for the non-beef livestock subject, or a product from the non-beef livestock subject, based on the traits. The model is typically a computer model. Values for the non-beef livestock subjects can be used to segregate the animals. Furthermore, various parameters that can be controlled during maintenance and growth of the non-beef livestock subjects can be input into the model in order to affect the way the animals are raised in order to obtain maximum value for the non-beef livestock subject when it is harvested.

In certain embodiments, meat or milk can be obtained at a time point that is affected by the inferred trait and one or more of the food intake, diet composition, and management of the non-beef livestock subject. For example, where the inferred trait of a non-beef livestock subject is high feed efficiency, which can be identified in quantitative or qualitative terms, meat or milk can be obtained at a time point that is sooner than a time point for a non-beef livestock subject with low feed efficiency. As another example, non-beef livestock subjects with different feed efficiencies can be separated, and those with lower feed efficiencies can be implanted with growth promotants or fed metabolic partitioning agents in order to maximize the profitability of a single non-beef livestock subject.

In another aspect, the present invention provides methods that allow effective measurement and sorting of animals individually, accurate and complete record keeping of genotypes and traits or characteristics for each animal, and production of an economic end point determination for each animal using growth performance data. Accordingly, the present invention provides a method for sorting non-beef livestock subjects. The method includes inferring a trait for both a first non-beef livestock subject and a second non-beef livestock subject from a nucleic acid sample of the first non-beef livestock subject and the second non-beef livestock subject. The inference is made by a method that includes identifying the nucleotide occurrence of at least one single nucleotide polymorphism (SNP), wherein the nucleotide occurrence is associated with the trait. The method further includes sorting the first non-beef livestock subject and the second non-beef livestock subject based on the inferred trait.

The method can further include measuring a physical characteristic of the first non-beef livestock subject and the second non-beef livestock subject, and sorting the first non-beef livestock subject and the second non-beef livestock subject based on both the inferred trait and the measured physical characteristic. The physical characteristic can be, for example, weight, breed, type or frame size, and can be measured using many methods known in the art.

In another aspect, the present invention provides methods that use analysis of non-beef livestock genetic variation to improve the genetics of the population to produce animals with consistent desirable characteristics, such as animals that yield a high percentage of lean meat and a low percentage of fat efficiently. Accordingly, in one aspect the present invention provides a method for selection and breeding of non-beef livestock subjects for a trait. The method includes inferring the genetic potential for a trait or a series of traits in a group of non-beef livestock candidates for use in breeding programs from a nucleic acid sample of the non-beef livestock candidates. The inference is made by a method that includes identifying the nucleotide occurrence of at least one single nucleotide polymorphism (SNP), wherein the nucleotide occurrence is associated with the trait or traits. Individuals are then selected from the group of candidates with a desired performance for the trait or traits for use in breeding programs. Progeny resulting from mating of selected parents would contain the optimum combination of traits, thus creating an enduring genetic pattern and line of animals with specific traits. These lines could be monitored for purity using the original SNP markers and could be identified from the entire population of non-beef livestock and protected from genetic theft.

In another aspect the present invention provides a method for cloning a non-beef livestock subject with a specific trait or series of traits. The method includes identifying nucleotide occurrences of at least one or at least two SNPs for the non-beef livestock subject, isolating a progenitor cell from the non-beef livestock subject, and generating a cloned non-beef livestock from the progenitor cell. The method can further include before identifying the nucleotide occurrences, identifying the trait of the non-beef livestock subject, wherein the non-beef livestock subject has a desired trait and wherein the at least one or at least two SNPs affect the trait.

Methods of cloning non-beef livestock are known in the art and can be used for the present invention. For example, methods of cloning pigs have been reported (See e.g., Carter D. B., et. al., “Phenotyping of transgenic cloned piglets,” Cloning Stem Cells 4: 131-45 (2002)).

For methods involving milk and dairy product traits, known methods for cloning cattle can be used (See e.g., Bondioli, “Commercial cloning of cattle by nuclear transfer”, In: Symposium on Cloning Mammals by Nuclear Transplantation, Seidel (ed), pp. 35-38, (1994); Willadsen, “Cloning of sheep and cow embryos,” Genome, 31: 956, (1989); Wilson et al., “Comparison of birth weight and growth characteristics of bovine calves produced by nuclear transfer (cloning), embryo transfer and natural mating”, Animal Reprod. Sci., 38: 73-83, (1995); and Barnes et al., “Embryo cloning in cattle: The use of in vitro matured oocytes”, J. Reprod. Fert., 97: 317-323, (1993)). These methods include somatic cell cloning (See e.g., Enright B. P. et al., “Reproductive characteristics of cloned heifers derived from adult somatic cells,” Biol. Reprod., 66: 291-6 (2002); Bruggerhoff K., et al., “Bovine somatic cell nuclear transfer using recipient oocytes recovered by ovum pick-up: effect of maternal lineage of oocyte donors,” Biol. Reprod., 66: 367-73 (2002); Wilmut, I., et al., “Somatic cell nuclear transfer,” Nature, 419: 583 (2002); Galli, C., et al., “Bovine embryo technologies,” Theriogenology, 59: 599 (2003); Heyman, Y., et al., “Novel approaches and hurdles to somatic cloning in cattle,” Cloning Stem Cells, 4: 47 (2002)).

This invention identifies animals that have superior traits, predicted very accurately, that can be used to identify parents of the next generation through selection. This invention provides a method for determining the optimum male and female parent to maximize the genetic components of dominance and epistasis thus maximizing heterosis and hybrid vigor in the market animals.

In another aspect, the present invention provides a non-beef livestock subject resulting from the selection and breeding aspect or the cloning aspect of the invention, discussed above.

In another aspect, the present invention provides a method of tracking a product of a non-beef livestock subject. The method includes identifying nucleotide occurrences for a series of genetic markers of the non-beef livestock subject, identifying the nucleotide occurrences for the series of genetic markers for a product sample, and determining whether the nucleotide occurrences of the non-beef livestock subject are the same as the nucleotide occurrences of the product sample. In this method identical nucleotide occurrences indicate that the product sample is from the non-beef livestock subject. The tracking method provides, for example, a method for historical and epidemiological tracking the location of an animal from embryo to birth through its growth period, to harvest and finally the retail product after the it has reached the consumer.

The series of genetic markers can be a series of single nucleotide polymorphisms (SNPs). The method can further include comparing the results of the above determination with a determination of whether the meat is from the non-beef livestock subject made using another tracking method. In this embodiment, the present invention provides quality control information that improves the accuracy of tracking the source of meat by a single method alone.

The nucleotide occurrence data for the non-beef livestock subject can be stored in a computer readable form, such as a database. Therefore, in one example, an initial nucleotide occurrence determination can be made for the series of genetic markers for a young non-beef livestock subject and stored in a database along with information identifying the non-beef livestock subject. Then, after meat from the non-beef livestock subject is obtained, possibly months or years after the initial nucleotide occurrence determination, and before and/or after the meat is shipped to a customer such as, for example, a wholesale distributor, a sample can be obtained from the product, meat, and nucleotide occurrence information determined using methods discussed herein. The database can then be queried using a user interface as discussed herein, with the nucleotide occurrence data from the meat sample to identify the non-beef livestock subject.

A series of markers or a series of SNPs as used herein, can include a series of at least 2, 3, 4, 5, 6, 7, 8, 9, 10, 15, 20, 25, 30, 35, 40, 45, 50, 75, 100, 150, 200, 250, 500, 1000, 2000, 2500, 5000, or 6000 markers, for example.

In another aspect, the present invention provides a method for diagnosing a health condition of a non-beef livestock subject. The method includes drawing an inference regarding a trait of the non-beef livestock subject for the health condition, from a nucleic acid sample of the subject. The inference is drawn by identifying, in the nucleic acid sample, at least one nucleotide occurrence of a single nucleotide polymorphism (SNP), wherein the nucleotide occurrence is associated with the trait.

The nucleotide occurrence of at least 2 SNPs can be determined. At least 2 SNPs can form a haploytpe, wherein the method identifies a haplotype allele that is associated with the trait. The method can include identifying a diploid pair of haplotype alleles for one or more haplotypes.

The health condition for this aspect of the invention, is resistance to disease or infection, susceptibility to infection with and shedding of pathogens such as E. Coli, Salmonella, Listeria, prion diseases and other organisms potentially pathogenic to humans, regulation of immune status and response to antigens, susceptibility to bloat, Johnes Disease, or liver abscess, previous exposure to infection or parasites, or health of respiratory and digestive tissues.

The present invention in another aspect provides a method for inferring a trait of a non-beef livestock subject from a nucleic acid sample of the subject, that includes identifying, in the nucleic acid sample, at least one nucleotide occurrence of a single nucleotide polymorphism (SNP). The nucleotide occurrence is associated with the trait, thereby allowing an inference of the trait.

These embodiments of the invention are based, in part, on a determination that single nucleotide polymorphisms (SNPs), including haploid or diploid SNPs, and haplotype alleles, including haploid or diploid haplotype alleles, allow an inference to be drawn as to the trait of a subject, particularly a non-beef livestock subject.

Accordingly, methods of the invention can involve determining the nucleotide occurrence of at least 2, 3, 4, 5, 10, 20, 30, 40, 50, etc. SNPs. The SNPs can form all or part of a haploytpe, wherein the method can identify a haplotype allele that is associated with the trait. Furthermore, the method can include identifying a diploid pair of haplotype alleles.

In another aspect, the present invention provides a method for identifying a non-beef livestock genetic marker that influences a trait. The method includes analyzing non-beef livestock genetic markers for association with the trait. The genetic marker can be a single nucleotide polymorphism (SNP), or can be at least two SNPs that influence the trait. Because the method can identify at least two SNPs, and in some embodiments, many SNPs, the method can identify not only additive genetic components, but non-additive genetic components such as dominance (i.e. dominating trait of an allele of one genomic over an allele of a another gene) and epistasis (i.e. interaction between genes at different loci). Furthermore, the method can uncover pleiotropic effects of SNP alleles (i.e. SNP alleles or haplotypes effects on many different traits), because many traits can be analyzed for their association with many SNPs using methods disclosed herein.

Nucleotide occurrences can be determined for essentially all, or all of the SNPs of a high-density, whole genome SNP map. This approach has the advantage over traditional approaches in that since it encompasses the whole genome, it identifies potential interactions of genomic products expressed from genes located anywhere on the genome without requiring preexisting knowledge regarding a possible interaction between the genomic products. An example of a high-density, whole genome SNP map is a map of at least about 1 SNP per 10,000 kb, at least 1 SNP per 500 kb or about 10 SNPs per 500 kb, or at least about 25 SNPs or more per 500 kb. Definitions of densities of markers may change across the genome and are determined by the degree of linkage disequilibrium within a genome region.

The invention includes methods for creating a high density map. The SNP markers and their surrounding sequence are compared to model organisms, for example human and mouse genomes, where the complete genomic sequence is known and syntenic regions identified or to a finished map of a species. The model organism map may serve as a template for ensuring complete coverage of the animal genome. The finished map has markers spaced in such a way to maximize the amount of linkage disequilibrium in a specific genetic region.

This map is used to mark all regions of the chromosomes in a single experiment utilizing thousands of experimental animals in an association study, to correlate genomic regions with complex and simple traits. These associations can be further analyzed to unravel complex interactions among genomic regions that contribute to the targeted trait or other traits, epistatic genetic interactions and pleiotropy. The invention of regional high density maps can also be used to identify targeted regions of chromosomes that influence traits.

Accordingly, in embodiments where SNPs that affect the same trait are identified that are located in different genes, the method can further include analyzing expression products of genes near the identified SNPs, to determine whether the expression products interact. As such, the present invention provides methods to detect epistatic genetic interactions. Laboratory methods are well known in the art for determining whether genomic products interact.

Where the trait is overall quality, the method can infer an overall average quality grade for a product obtained from the non-beef livestock subject. Alternatively, the method can infer the best or the worst quality grade expected for a product obtained from the non-beef livestock subject. Additionally, as indicated above, the trait can be a characteristic used to classify the product.

The methods of the present invention that infer a trait can be used in place of present methods used to determine the trait, or can be used to further substantiate a classification of meat or another product using present methods.

In aspects of the present invention directed at identifying a non-beef livestock genetic marker that influences a trait, present methods for determining a trait, such as a characteristic of pork, can be used in the methods to identify an association between a genetic marker, typically at least one SNP or haplotype, with a trait. For example, DNA samples from non-beef livestock subjects can be obtained, and nucleotide occurrences for at least one SNP in the DNA samples can be determined. Traditional methods can be used to determine the trait. As will be understood, statistical methods can then be used to identify associations between the nucleotide occurrences and the trait. Accordingly, methods of the present invention enables a correlation between carcass value and genetic variation, so as to help identify superior genetic types for future breeding or cloning and management purposes, and to identify management practices that will maximize the value of the arrival in the market.

Where the trait is pork tenderness, for example, methods of the present invention can infer from a sample of a non-beef livestock subject, such as a live non-beef livestock subject, whether pork if cooked properly, would be tender. The method can be used in place of current post-methods.

In another aspect, the present invention provides a method for identifying a non-beef livestock genomic associated with a trait. The method includes identifying a non-beef livestock single nucleotide polymorphism (SNP) that influences a trait by analyzing a genome-wide non-beef livestock SNP map for association with the trait, wherein the SNP is found on a target region of a non-beef livestock chromosome. Genes present on the target region are then identified. The presence of a genomic on the target region of the non-beef livestock chromosome indicates that the genomic is a candidate genomic for association with the trait. The candidate genomic can then be analyzed using methods known in the art to determine whether it is associated with the trait.

In another aspect, the present invention provides a method for identifying a breed of a non-beef livestock subject. The method includes identifying a nucleotide occurrence of a non-beef livestock single nucleotide polymorphism (SNP) from a nucleic acid sample of the subject, wherein the nucleotide occurrence is associated with the breed of the subject. The method typically includes identifying nucleotide occurrences of at least two SNPs from the nucleic acid sample, wherein the nucleotide occurrences are associated with the breed of the subject.

SNP that identifies a breed, by analyzing a genome-wide non-beef livestock SNP map for association with the trait, wherein the SNP is found on a target region of a non-beef livestock chromosome. Genes present on the target region are then identified. The presence of a genomic on the target region of the non-beef livestock chromosome indicates that the genomic is a candidate genomic for association with the trait. The candidate genomic can then be analyzed using methods known in the art to determine whether it is associated with the trait.

In another aspect, the present invention provides a high-throughput system for determining the nucleotide occurrences at a series of non-beef livestock single nucleotide polymorphisms (SNPs). The system typically includes a hybridization medium comprising a series of oligonucleotides, which is typically one of the following: a solid support to which a series of oligonucleotides can be directly or indirectly attached, a homogeneous assay or a microfluidic device. Each of these hybridization mediums is used to determine the nucleotide occurrence of non-beef livestock SNPs that are associated with a trait.

Accordingly, the oligonucleotides are used to determine the nucleotide occurrence of non-beef livestock SNPs that are associated with a trait. The determination can be made by selecting oligonucleotides that bind at or near a genomic location of each SNP of the series of non-beef livestock SNPs. For example, such oligonucleotides include forward and reverse oligonucleotides that can support amplification of the sequences provided in Table 1 (SEQ ID NOs:1-96,631). Additional oligonucleotides would include extension primers that hybridize in proximity to an SNP provided in SEQ ID NOs:96,631 and support extension to the SNP for purposes of identification. The high-throughput system of the present invention typically includes a reagent handling mechanism that can be used to apply a reagent, typically a liquid, to the solid support. The binding of an oligonucleotide of the series of oligonucleotides to a polynucleotide isolated from a genome can be affected by the nucleotide occurrence of the SNP. The high-throughput system can include a mechanism effective for moving a solid support and a detection mechanism. The detection method detects binding or tagging of the oligonucleotides.

High-throughput systems for analyzing SNPs, known in the art such as the UHT SNP-IT platform (Orchid Biosciences, Princeton, N.J.) MassArray™ system (Sequenom, San Diego, Calif.) and the integrated SNP genotyping system available from Illumina (San Diego, Calif.), TaqMan™ (ABI, Foster City, Calif.) can be used with the present invention. However, the present invention provides a high-throughput system that is designed to detect nucleotide occurrences of non-beef livestock SNPs, or a series of non-beef livestock SNPs that can make up a series of haplotypes. Therefore, as indicated above the system includes a solid support or other method to which a series of oligonucleotides can be associated that are used to determine a nucleotide occurrence of a SNP for a series of non-beef livestock SNPs that are associated with a trait. The system can further include a detection mechanism for detecting binding the series of oligonucleotides to the series of SNPs. Such detection mechanisms are known in the art.

The high-throughput system can be a microfluidics device. Numerous microfluidic devices are known that include solid supports with microchannels (See e.g., U.S. Pat. Nos. 5,304,487, 5,110,745, 5,681,484, and 5,593,838).

The high-throughput systems of the present invention are designed to determine nucleotide occurrences of one SNP or a series of SNPs. The systems can determine nucleotide occurrences of an entire genome-wide high-density SNP map.

Numerous methods are known in the art for determining the nucleotide occurrence for a particular SNP in a sample. Such methods can utilize one or more oligonucleotide probes or primers, including, for example, an amplification primer pair, that selectively hybridize to a target polynucleotide, which corresponds to one or more non-beef livestock SNP positions, such as those provided in Table 1 (SEQ ID NOs:1-96,631). Oligonucleotide probes useful in practicing a method of the invention can include, for example, an oligonucleotide that is complementary to and spans a portion of the target polynucleotide, including the position of the SNP, wherein the presence of a specific nucleotide at the position (i.e., the SNP) is detected by the presence or absence of selective hybridization of the probe. Such a method can further include contacting the target polynucleotide and hybridized oligonucleotide with an endonuclease, and detecting the presence or absence of a cleavage product of the probe, depending on whether the nucleotide occurrence at the SNP site is complementary to the corresponding nucleotide of the probe.

An oligonucleotide ligation assay also can be used to identify a nucleotide occurrence at a polymorphic position, wherein a pair of probes that selectively hybridize upstream and adjacent to and downstream and adjacent to the site of the SNP, and wherein one of the probes includes a terminal nucleotide complementary to a nucleotide occurrence of the SNP. Where the terminal nucleotide of the probe is complementary to the nucleotide occurrence, selective hybridization includes the terminal nucleotide such that, in the presence of a ligase, the upstream and downstream oligonucleotides are ligated. As such, the presence or absence of a ligation product is indicative of the nucleotide occurrence at the SNP site.

An oligonucleotide also can be useful as a primer, for example, for a primer extension reaction, wherein the product (or absence of a product) of the extension reaction is indicative of the nucleotide occurrence. In addition, a primer pair useful for amplifying a portion of the target polynucleotide including the SNP site can be useful, wherein the amplification product is examined to determine the nucleotide occurrence at the SNP site. Particularly useful methods include those that are readily adaptable to a high throughput format, to a multiplex format, or to both. The primer extension or amplification product can be detected directly or indirectly and/or can be sequenced using various methods known in the art. Amplification products which span a SNP loci can be sequenced using traditional sequence methodologies (e.g., the “dideoxy-mediated chain termination method,” also known as the “Sanger Method” (Sanger, F., et al., J. Molec. Biol. 94: 441 (1975); Prober et al. Science 238: 336-340 (1987)) and the “chemical degradation method,” “also known as the “Maxam-Gilbert method” (Maxam, A. M., et al., Proc. Natl. Acad. Sci. (U.S.A.) 74: 560 (1977)), both references herein incorporated by reference) to determine the nucleotide occurrence at the SNP loci.

Methods of the invention can identify nucleotide occurrences at SNPs using genome-wide sequencing or “microsequencing” methods. Whole-genome sequencing of individuals identifies all SNP genotypes in a single analysis. Microsequencing methods determine the identity of only a single nucleotide at a “predetermined” site. Such methods have particular utility in determining the presence and identity of polymorphisms in a target polynucleotide. Such microsequencing methods, as well as other methods for determining the nucleotide occurrence at a SNP loci are discussed in Boyce-Jacino, et al., U.S. Pat. No. 6,294,336, incorporated herein by reference, and summarized herein.

Microsequencing methods include the Genetic Bit Analysis method disclosed by Goelet, P. et al. (WO 92/15712, herein incorporated by reference). Additional, primer-guided, nucleotide incorporation procedures for assaying polymorphic sites in DNA have also been described (Komher, J. S. et al, Nucl. Acids. Res. 17: 7779-7784 (1989); Sokolov, B. P., Nucl. Acids Res. 18: 3671 (1990); Syvanen, A.-C., et al., Genomics 8: 684-692 (1990); Kuppuswamy, M. N. et al., Proc. Natl. Acad. Sci. (U.S.A.) 88: 1143-1147 (1991); Prezant, T. R. et al, Hum. Mutat. 1: 159-164 (1992); Ugozzoli, L. et al., GATA 9: 107-112 (1992); Nyren, P. et al., Anal. Biochem. 208: 171-175 (1993); and Wallace, WO89/10414). These methods differ from Genetic Bit™. Analysis in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al. Amer. J. Hum. Genet. 52: 46-59 (1993)).

Alternative microsequencing methods have been provided by Mundy, C. R. (U.S. Pat. No. 4,656,127) and Cohen, D. et al (French Patent 2,650,840; PCT Appln. No. WO91/02087) which discusses a solution-based method for determining the identity of the nucleotide of a polymorphic site. As in the Mundy method of U.S. Pat. No. 4,656,127, a primer is employed that is complementary to allelic sequences immediately 3′-to a polymorphic site.

In response to the difficulties encountered in employing gel electrophoresis to analyze sequences, alternative methods for microsequencing have been developed. Macevicz (U.S. Pat. No. 5,002,867), for example, describes a method for determining nucleic acid sequence via hybridization with multiple mixtures of oligonucleotide probes. In accordance with such method, the sequence of a target polynucleotide is determined by permitting the target to sequentially hybridize with sets of probes having an invariant nucleotide at one position, and variant nucleotides at other positions. The Macevicz method determines the nucleotide sequence of the target by hybridizing the target with a set of probes, and then determining the number of sites that at least one member of the set is capable of hybridizing to the target (i.e., the number of “matches”). This procedure is repeated until each member of a sets of probes has been tested.

Boyce-Jacino, et al., U.S. Pat. No. 6,294,336 provides a solid phase sequencing method for determining the sequence of nucleic acid molecules (either DNA or RNA) by utilizing a primer that selectively binds a polynucleotide target at a site wherein the SNP is the most 3′ nucleotide selectively bound to the target.

Oliphant et al. report a method that utilizes BeadArray™ Technology that can be used in the methods of the present invention to determine the nucleotide occurrence of a SNP. (supplement to Biotechniques, June 2002). Additionally, nucleotide occurrences for SNPs can be determined using a DNAMassARRAY system (SEQUENOM, San Diego, Calif.). This system combines proprietary SpectroChips™, microfluidics, nanodispensing, biochemistry, and MALDI-TOF MS (matrix-assisted laser desorption ionization time of flight mass spectrometry).

As another example, the nucleotide occurrences of non-beef livestock SNPs in a sample can be determined using the SNP-IT™ method (Orchid BioSciences, Inc., Princeton, N.J.). In general, SNP-IT™ is a 3-step primer extension reaction. In the first step a target polynucleotide is isolated from a sample by hybridization to a capture primer, which provides a first level of specificity. In a second step the capture primer is extended from a terminating nucleotide trisphosphate at the target SNP site, which provides a second level of specificity. In a third step, the extended nucleotide trisphosphate can be detected using a variety of known formats, including: direct fluorescence, indirect fluorescence, an indirect colorimetric assay, mass spectrometry, fluorescence polarization, etc. Reactions can be processed in 384 well format in an automated format using a SNPstream™ instrument ((Orchid BioSciences, Inc., Princeton, N.J.). Other formats include TaqMan™, Rolling circle, Fluorescent polarization, etc.

Accordingly, using the methods described above, the non-beef livestock haplotype allele or the nucleotide occurrence of a non-beef livestock SNP can be identified using an amplification reaction, a primer extension reaction, or an immunoassay. The non-beef livestock haplotype allele or non-beef livestock SNP can also be identified by contacting polynucleotides in the sample or polynucleotides derived from the sample, with a specific binding pair member that selectively hybridizes to a polynucleotide region comprising the non-beef livestock SNP, under conditions wherein the binding pair member specifically binds at or near the non-beef livestock SNP. The specific binding pair member can be an antibody or a polynucleotide.

The nucleotide occurrence of a SNP can be identified by other methodologies as well as those discussed above. For example, the identification can use microarray technology, which can be performed with or without PCR, or sequencing methods such as mass spectrometry, scanning electron microscopy, or methods in which a polynucleotide flows past a sorting device that can detect the sequence of the polynucleotide.

The high-throughput systems of the present invention typically utilize selective hybridization. As used herein, the term “selective hybridization” or “selectively hybridize,” refers to hybridization under moderately stringent or highly stringent conditions such that a nucleotide sequence preferentially associates with a selected nucleotide sequence over unrelated nucleotide sequences to a large enough extent to be useful in identifying a nucleotide occurrence of a SNP. It will be recognized that some amount of non-specific hybridization is unavoidable, but is acceptable provide that hybridization to a target nucleotide sequence is sufficiently selective such that it can be distinguished over the non-specific cross-hybridization, for example, at least about 2-fold more selective, generally at least about 3-fold more selective, usually at least about 5-fold more selective, and particularly at least about 10-fold more selective, as determined, for example, by an amount of labeled oligonucleotide that binds to target nucleic acid molecule as compared to a nucleic acid molecule other than the target molecule, particularly a substantially similar (i.e., homologous) nucleic acid molecule other than the target nucleic acid molecule. Conditions that allow for selective hybridization can be determined empirically, or can be estimated based, for example, on the relative GC:AT content of the hybridizing oligonucleotide and the sequence to which it is to hybridize, the length of the hybridizing oligonucleotide, and the number, if any, of mismatches between the oligonucleotide and sequence to which it is to hybridize (see, for example, Sambrook et al., “Molecular Cloning: A laboratory manual (Cold Spring Harbor Laboratory Press 1989)).

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

The term “polynucleotide” is used broadly herein to mean a sequence of deoxyribonucleotides or ribonucleotides that are linked together by a phosphodiester bond. For convenience, the term “oligonucleotide” is used herein to refer to a polynucleotide that is used as a primer or a probe. Generally, an oligonucleotide useful as a probe or primer that selectively hybridizes to a selected nucleotide sequence is at least about 15 nucleotides in length, usually at least about 18 nucleotides, and particularly about 21 nucleotides or more in length.

A polynucleotide can be RNA or can be DNA, which can be a genomic or a portion thereof, a cDNA, a synthetic polydeoxyribonucleic acid sequence, or the like, and can be single stranded or double stranded, as well as a DNA/RNA hybrid. In various embodiments, a polynucleotide, including an oligonucleotide (e.g., a probe or a primer) can contain nucleoside or nucleotide analogs, or a backbone bond other than a phosphodiester bond. In general, the nucleotides comprising a polynucleotide are naturally occurring deoxyribonucleotides, such as adenine, cytosine, guanine or thymine linked to 2′-deoxyribose, or ribonucleotides such as adenine, cytosine, guanine or uracil linked to ribose. However, a polynucleotide or oligonucleotide also can contain nucleotide analogs, including non-naturally occurring synthetic nucleotides or modified naturally occurring nucleotides. Such nucleotide analogs are well known in the art and commercially available, as are polynucleotides containing such nucleotide analogs (Lin et al., Nucl. Acids Res. 22: 5220-5234 (1994); Jellinek et al., Biochemistry 34: 11363-11372 (1995); Pagratis et al., Nature Biotechnol. 15: 68-73 (1997), each of which is incorporated herein by reference).

The covalent bond linking the nucleotides of a polynucleotide generally is a phosphodiester bond. However, the covalent bond also can be any of numerous other bonds, including a thiodiester bond, a phosphorothioate bond, a peptide-like bond or any other bond known to those in the art as useful for linking nucleotides to produce synthetic polynucleotides (see, for example, Tam et al., Nucl. Acids Res. 22: 977-986 (1994); Ecker and Crooke, BioTechnology 13: 351360 (1995), each of which is incorporated herein by reference). The incorporation of non-naturally occurring nucleotide analogs or bonds linking the nucleotides or analogs can be particularly useful where the polynucleotide is to be exposed to an environment that can contain a nucleolytic activity, including, for example, a tissue culture medium or upon administration to a living subject, since the modified polynucleotides can be less susceptible to degradation.

A polynucleotide or oligonucleotide comprising naturally occurring nucleotides and phosphodiester bonds can be chemically synthesized or can be produced using recombinant DNA methods, using an appropriate polynucleotide as a template. In comparison, a polynucleotide or oligonucleotide comprising nucleotide analogs or covalent bonds other than phosphodiester bonds generally are chemically synthesized, although an enzyme such as T7 polymerase can incorporate certain types of nucleotide analogs into a polynucleotide and, therefore, can be used to produce such a polynucleotide recombinantly from an appropriate template (Jellinek et al., supra, 1995). Thus, the term polynucleotide as used herein includes naturally occurring nucleic acid molecules, which can be isolated from a cell, as well as synthetic molecules, which can be prepared, for example, by methods of chemical synthesis or by enzymatic methods such as by the polymerase chain reaction (PCR).

In various embodiments for identifying nucleotide occurrences of SNPs, it can be useful to detectably label a polynucleotide or oligonucleotide. Detectable labeling of a polynucleotide or oligonucleotide is well known in the art. Particular non-limiting examples of detectable labels include chemiluminescent labels, fluorescent labels, radiolabels, enzymes, haptens, or even unique oligonucleotide sequences.

A method of the identifying a SNP also can be performed using a specific binding pair member. As used herein, the term “specific binding pair member” refers to a molecule that specifically binds or selectively hybridizes to another member of a specific binding pair. Specific binding pair member include, for example, probes, primers, polynucleotides, antibodies, etc. For example, a specific binding pair member includes a primer or a probe that selectively hybridizes to a target polynucleotide that includes a SNP loci, or that hybridizes to an amplification product generated using the target polynucleotide as a template. Generally binding pair members include forward and reverse primers that can amplify a target sequence that includes, for example, any one of SEQ ID NOs:1-96,631.

As used herein, the term “specific interaction,” or “specifically binds” or the like means that two molecules form a complex that is relatively stable under physiologic conditions. The term is used herein in reference to various interactions, including, for example, the interaction of an antibody that binds a polynucleotide that includes a SNP site; or the interaction of an antibody that binds a polypeptide that includes an amino acid that is encoded by a codon that includes a SNP site. According to methods of the invention, an antibody can selectively bind to a polypeptide that includes a particular amino acid encoded by a codon that includes a SNP site. Alternatively, an antibody may preferentially bind a particular modified nucleotide that is incorporated into a SNP site for only certain nucleotide occurrences at the SNP site, for example using a primer extension assay.

A specific interaction can be characterized by a dissociation constant of at least about 1×10⁻⁶ M, generally at least about 1×10⁻⁷ M, usually at least about 1×10⁻⁸ M, and particularly at least about 1×10⁻⁹ M or 1×10⁻¹⁰ M or greater. A specific interaction generally is stable under physiological conditions, including, for example, conditions that occur in a living individual such as a human or other vertebrate or invertebrate, as well as conditions that occur in a cell culture such as used for maintaining mammalian cells or cells from another vertebrate organism or an invertebrate organism. Methods for determining whether two molecules interact specifically are well known and include, for example, equilibrium dialysis, surface plasmon resonance, and the like.

The present invention also provides a method for selecting a pig for use in xenotransplation. The method includes inferring a trait of a non-beef livestock subject from a nucleic acid sample of the subject, by identifying in the nucleic acid sample, at least one nucleotide occurrence of a single nucleotide polymorphism (SNP). The nucleotide occurrence is associated with the trait. For these embodiments, the trait is the suitability of organs of the pig for transplantation into human transplantation. Organs that can be used for transplantation include, but are not limited to, whole organs such as hearts, kidney, liver, and pancreas.

The invention also relates to kits, which can be used, for example, to perform a method of the invention. Thus, in one embodiment, the invention provides a kit for identifying nucleotide occurrences or haplotype alleles of non-beef livestock SNPs. Such a kit can contain, for example, an oligonucleotide probe, primer, or primer pair, or combinations thereof. Such oligonucleotides being useful, for example, to identify a SNP or haplotype allele as disclosed herein; or can contain one or more polynucleotides corresponding to a portion of a non-beef livestock genomic containing one or more nucleotide occurrences associated with a non-beef livestock trait, such polynucleotide being useful, for example, as a standard (control) that can be examined in parallel with a test sample. In addition, a kit of the invention can contain, for example, reagents for performing a method of the invention, including, for example, one or more detectable labels, which can be used to label a probe or primer or can be incorporated into a product generated using the probe or primer (e.g., an amplification product); one or more polymerases, which can be useful for a method that includes a primer extension or amplification procedure, or other enzyme or enzymes (e.g., a ligase or an endonuclease), which can be useful for performing an oligonucleotide ligation assay or a mismatch cleavage assay; and/or one or more buffers or other reagents that are necessary to or can facilitate performing a method of the invention. The primers or probes can be included in a kit in a labeled form, for example with a label such as biotin or an antibody.

In one embodiment, a kit of the invention provides a plurality of oligonucleotides of the invention, including one or more oligonucleotide probes or one or more primers, including forward and/or reverse primers, or a combination of such probes and primers or primer pairs. Such a kit also can contain probes and/or primers that conveniently allow a method of the invention to be performed in a multiplex format.

The kit can also include instructions for using the probes or primers to determine a nucleotide occurrence of at least one non-beef livestock SNPs.

In another aspect, the present invention provides a computer system that includes a database having records containing information regarding a series of non-beef livestock single nucleotide polymorphisms (SNPs), and a user interface allowing a user to input nucleotide occurrences of the series of non-beef livestock SNPs for a non-beef livestock subject. The user interface can be used to query the database and display results of the query. The database can include records representing some or all of the SNP of a non-beef livestock SNP map, such as a high-density non-beef livestock SNP map. The database can also include information regarding haplotypes and haplotype alleles from the SNPs. Furthermore, the database can include information regarding traits and/or traits that are associated with some or all of the SNPs and/or haplotypes. In these embodiments the computer system can be used, for example, for any of the aspects of the invention that infer a trait of a non-beef livestock subject.

The computer system of the present invention can be a stand-alone computer, a conventional network system including a client/server environment and one or more database servers, and/or a handheld device. A number of conventional network systems, including a local area network (LAN) or a wide area network (WAN), are known in the art. Additionally, client/server environments, database servers, and networks are well documented in the technical, trade, and patent literature. For example, the database server can run on an operating system such as UNIX, running a relational database management system, a World Wide Web application, and a World Wide Web Server. When the computer system is a handheld device it can be a personal digital assistant (PDA) or another type of handheld device, of which many are known.

Typically, the database of the computer system of the present invention includes information regarding the location and nucleotide occurrences of non-beef livestock SNPs. Information regarding genomic location of the SNP can be provided for example by including sequence information of consecutive sequences surrounding the SNP, that only 1 part of the genome provides 100% match, or by providing a position number of the SNP with respect to an available sequence entry, such as a Genbank sequence entry, or a sequence entry for a private database, or a commercially licensed database of DNA sequences. The database can also include information regarding nucleotide occurrences of SNPs, since as discussed herein typically nucleotide occurrences of less than all four nucleotides occur for a SNP.

The database can include other information regarding SNPs or haplotypes such as information regarding frequency of occurrence in a non-beef livestock population. Furthermore, the database can be divided into multiple parts, one for storing sequences and the others for storing information regarding the sequences. The database may contain records representing additional information about a SNP, for example information identifying the genomic in which a SNP is found, or nucleotide occurrence frequency information, or characteristics of the library or clone which generated the DNA sequence, or the relationship of the sequence surrounding the SNP to similar DNA sequences in other species.

The parts of the database of the present invention can be flat file databases or relational databases or object-oriented databases. The parts of the database can be internal databases, or external databases that are accessible to users. An internal database is a database maintained as a private database, typically maintained behind a firewall, by an enterprise. An external database is located outside an internal database, and is typically maintained by a different entity than an internal database. A number of external public biological sequence databases, particularly SNP databases, are available and can be used with the current invention. For example, the dbSNP database available from the National Center for Biological Information (NCBI), part of the National Library of Medicine, can be used with the current invention to provide comparative genomic information to assist in identifying non-beef livestock SNPs.

In another aspect, the current invention provides a population of information regarding non-beef livestock SNPs and haplotypes. The population of information can include an identification of traits associated with the SNPs and haplotyopes. The population of information is typically included within a database, and can be identified using the methods of the current invention. The population of sequences can be a subpopulation of a larger database, that contains only SNPs and haplotypes related to a particular trait. For example, the subpopulation can be identified in a table of a relational database. A population of information can include all of the SNPs and/or haplotypes of a genome-wide SNP map.

In addition to the database discussed above, the computer system of the present invention includes a user interface capable of receiving entry of nucleotide occurrence information regarding at least one SNP. The interface can be a graphic user interface where entries and selections are made using a series of menus, dialog boxes, and/or selectable buttons, for example. The interface typically takes a user through a series of screens beginning with a main screen. The user interface can include links that a user may select to access additional information relating a non-beef livestock SNP map.

The function of the computer system of the present invention that carries out the trait inference methods typically includes a processing unit that executes a computer program product, itself representing another aspect of the invention, that includes a computer-readable program code embodied on a computer-usable medium and present in a memory function connected to the processing unit. The memory function can be ROM or RAM.

The computer program product, itself another aspect of the invention, is read and executed by the processing unit of the computer system of the present invention, and includes a computer-readable program code embodied on a computer-usable medium. The computer-readable program code relates to a plurality of sequence records stored in a database. The sequence records can contain information regarding the relationship between nucleotide occurrences of a series of non-beef livestock single nucleotide polymorphisms (SNPs) and a trait of one or more traits. The computer program product can include computer-readable program code for providing a user interface capable of allowing a user to input nucleotide occurrences of the series of non-beef livestock SNPs for a non-beef livestock subject, locating data corresponding to the entered query information, and displaying the data corresponding to the entered query. Data corresponding to the entered query information is typically located by querying a database as described above.

In another embodiment of the present invention, the computer system and computer program products are used to perform bioeconomic valuations used to perform methods described herein, such as methods for estimating the value of a non-beef livestock subject or a product obtained therefrom.

Certain embodiments of the present invention provide methods, systems, and kits identical to those discussed above, and herein, except that the trait is milk production, a trait affecting milk production, a characteristic of milk, a characteristic of a dairy product, milk component composition, or mastitis resistance. In these embodiments, the methods, systems, and kits relate to all livestock (i.e. they include beef livestock).

Accordingly, in certain embodiments, the present invention provides a method for inferring from a nucleic acid sample of a livestock, a trait of milk production, a trait affecting milk production, a characteristic of milk, a characteristic of a dairy product, a milk component composition including fat, protein, and bioreactive molecules, or mastitis resistance, for the livestock. The method includes identifying in the nucleic acid sample, at least one nucleotide occurrence of a single nucleotide polymorphism (SNP), wherein the nucleotide occurrence is associated with the trait and wherein the trait is thereby inferred.

The livestock subject can be, for example, a cow, a goat, a sheep, a buffalo, a camel, a horse, or a deer. The trait can be, for example, milk protein content, milk fat content, milk amino acid profile, milk fatty acid profile, bioreactive molecule content, milk taste appeal, or taste appeal of a dairy product. Furthermore, the trait can be taste appeal of milk, cheese, yogurt, cream, butter, or ice cream. Alternatively, the trait can be milk or dairy product solids content, calcium content, riboflavin content, nitrogen potassium content, protein content, casein content, fat content, whey content, vitamin A content, vitamin D content, or phosphorus content. The trait can also be lactation period or production in milk of a transgenic protein or transgenically-produced pharmaceutical product.

### EXAMPLE

Approximately 1×coverage of the chicken genome was sequenced (MMIC) to identify SNP markers. Genomic DNA libraries from four (4) lines of chickens comprising a dam-line broiler, a sire-line broiler, a commercial layer, and Red Jungle Fowl were created using strategies developed by Celera Genomics (Venter et al. 2001. Science 291: 1145-1434). The constructed libraries were size selected to create 3 distinct categories for whole-genome shotgun sequencing: two point five (2.5), ten (10) and fifty (50) kilobase insert libraries.

The two point five (2.5) kb libraries were sequenced producing fragments of over 600 bp. The number of fragments of each source of sequence was: dam-line broiler—418,299, sire-line broiler—436,522, layer—444,423, and Red Jungle Fowl—464,224, for a total of 1,095,014,051 bp of sequence.

The fragments were aligned using proprietary assembly programs developed by Celera Genomics and single nucleotide polymorphisms (SNPs) identified by mismatches of the genomic sequence at a single base. There were 96,631 fragments (see SEQ ID NOs:1-96,631 included in Table 1 on a compact disc as filed herewith) identified with single nucleotide differences or SNPs, or a putative SNP marker was identified approximately every 11,000 bases. The frequency of each base transition or substitution follows the distribution of human and cattle SNP data: G to A—35.5%, T to C—35.7%, G to T—7.1%, G to C—6.9%, A to C—7.3%, and A to T—7.6%.

To map MMIC sequence and develop an evenly dispersed informative map for discovery of chicken traits, public working draft chicken sequences (e.g., the world wide web at http://genome.wustl.edu/projects/chicken/ and http://www.genome.gov/11510730) were downloaded from Washington University Medical School Genome Center Website (e.g., world wide web at http://genome.wustl.edu/projects/chicken/index.php?softmask=1). All fragments from MMIC were repeat-masked then blasted to the public chicken genome working draft. The present study has determined that 95.6% of MMIC fragments have homology with e values less than 10⁻⁵.

A Whole-Genome Chicken Discovery Map (WGCDM) is developed by selecting 8,000 SNP markers from the 96,631 putative markers (SEQ ID NOs:1-96,631). Each marker will be separated by approximately 150,000 bp, approximating a 0.5 cM discovery map. An exemplary model for developing such a map is provided in PCT Application No. PCT/US2003/04176, filed Dec. 31, 2003, incorporated herein by reference. Approximately 12 putative SNP markers are available for selection for WGCDM within each 150,000 bp bin of chicken sequence. Other factors such as location to coding regions, homology to other species, actual nucleotide distribution, and assay development potential will be considered when selecting SNP markers to undergo validation to create the discovery map.

With regard to selecting an experimental population, birds or avian species from a commercial production or breeding facility are selected for study. Each bird or avian species must have any or all of the following production phenotypic traits recorded: egg production, feed efficiency, livability, meat yield, longevity, white meat yield, dark meat yield, disease resistance, disease susceptibility, optimal diet time to maturity, time to a target weight, weight at a target time point, average daily weight gain, meat quality, muscle content, fat content, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, or conception rate. Birds may also have the following health information: general robust health and/or specific resistance to any infectious or genetic disease, including, but not limited to Exotic New Castle Disease, Salmonella infection, ascites, and Listeria infection.

The population structure can be of several types. For a linkage-disequilibrium (LD) study, known as a population-based design, one possible experimental design would utilize 3000 unrelated commercial birds or avian species will be phenotypically characterized for the traits described above. For a study that relies on linkage-disequilibrium and linkage analysis, known as a family-based design, one possible experimental design would contain 2000 progeny from 40 sires, mated to 2000 dams, with half-sib groups of 50 progeny per sire. Other designs are possible depending upon the use of the results.

The present disclosure provides 96,631 putative markers (SEQ ID NOs:1-96,631) identified from whole-genome shotgun sequencing and assembly. Using in silico techniques, approximately 8,000 of these markers are selected to undergo a marker validation test. Each of the putative discovery markers are tested in a small validation group of 24 to 40 animals, depending upon the experimental population. Markers failing assay development, Mendelian inheritance checks, Hardy-Weinberg equilibrium, monomorphic tests or paralog tests are replaced with other markers within the 150,000 bp bins of genomic sequence until a complete map of at least 8,000 evenly dispersed markers are identified to create the WGCDM.

A whole-genome association study can be undertaken in a number of ways depending on the number of animals, number of traits under study and utility of the product. The most likely, but not only, design comprises genotyping individual animals with the WGCDM markers. The results are platform independent and would result in a genotype such as XX, XY or YY for each animal at each SNP locus.

Another exemplary strategy includes pooling nucleic acids from about the top 10% and about the bottom 10% of animals based on the value of their trait. These pooled nucleic acid samples can be genotyped using quantitative PCR methods to determine the relative distribution of each nucleotide in the sample. Differences in the estimates of allele frequency of the high and low groups can be used to triage the markers and identify those that are associated with the traits of interest. When the target markers are identified, all animals can be genotyped with the markers.

The analysis of whole-genome data is also included in the present study. Exemplary analysis techniques can be divided generally into those techniques relating to 1) population-based designs and 2) family-based designs.

With regard to population-based designs, the simplest and most conservative approach is to perform least-squares regression for every SNP. The input to the analysis is whether there are zero, one or two occurrences of a certain allele. The null hypothesis of no association is tested using a test statistic such as the regression variance (F) ratio. Two parameters are estimated, the significance of a marker on phenotype and the size of the effect. When testing hundreds of SNPs in a single experiment, the probability of falsely identifying significant markers (false positives) is very high and results must be adjusted for the effect. Adjustments to the significance thresholds include: the Bonferroni correction, the Lander and Botstein (Nature Genetics. 1995. 11(3): 241-247.) genome-wide significance thresholds, or permutation tests (Churchill and Doerge. 1994. Genetics 138: 963-971). However, the overestimation of the size of the allelic effects is a serious problem when performing regressions at individual SNP. This occurs because of the co-linearity of the SNP genotype data. Simultaneous estimation of all allelic effects using least-squares regression is not possible. Because data sets are of limited size, there will be insufficient degrees of freedom to fit all effects in the one regression model.

Xu (Genetics 163: 789-801, 2003) describes a Bayesian regression model that can be used to simultaneously estimate allelic effects of all SNP in a genetic association study. This method utilizes shrinkage parameters that can be estimated from the data. However, the method has no formal means to set significance thresholds, it only highlights which SNP have negligible effect. To overcome this problem, the Bayesian regression model described previously could be used in conjunction with a variable selection procedure (George and McCulloch, Journal of the American Statistical Association 88: 881-889, 1993) or Bayesian model averaging could be utilized as reviewed in a paper by Hoeting et. al. (Statistical Science 14, 382-401, 1999).

However, least-squares and Bayesian regression still treat each SNP as independent, whether SNP are tested individually, or simultaneously in one analysis. If SNP markers are correlated due to proximity of the chromosome, these strategies can be inefficient. Several methods have emerged which analyze an ordered set of genetic markers known as a haplotype block or a chromosome segment. For each block an individual will carry a maternal and paternal haplotype. One approach to analyzing haplotype blocks is to fit the maternal and paternal haplotypes of each animal using either a standard linear model framework or using Bayesian analysis. The Bayesian analysis will be able to handle the situation of analyzing many blocks simultaneously. Meuwissen et al. (Genetics 157: 1819-1829) simulated the effects of 50,000 marker haplotypes. In a Bayesian analysis they were able to estimate all haplotype effects using only 2,200 observations. Shrinkage parameters, similar to that used by Xu (see above) were used to estimate the approximate significance of each segment.

Methods are also emerging which identify the minimal set of SNPs, called tagSNPs, which are able to resolve all possible haplotypes for a given region (Stram et. al. 2003 Human Heredity 55: 27-36) by selecting a maximally informative set of single-nucleotide polymorphisms for association analyses using linkage disequilibrium (Carlson et. al. 2004. American Journal of Human Genetics 74: 106-120).

When a causal mutation affecting a quantitative trait occurs on a chromosome, the mutation is initially in complete linkage disequilibrium with all other alleles on the chromosome, but not necessarily across all individuals in the population. The disequilibrium among distant alleles erodes quickly due to recombination, but erosion is slower for alleles that are close. If individuals in a population share the same causal mutation they will also likely share the same alleles proximate to it. The assumption here is that they share a distant unknown common ancestor. Haplotype block analysis methods can be further improved by accounting for the degree of haplotype sharing among individuals. Sharing can be more accurately defined as the degree to which haplotypes are identical by descent (IBD). Meuwissen and Goddard (2000. Genetics 155: 421-30) have proposed using a variance covariance matrix of haplotype effects in the model. The covariances between haplotype effects are the probabilities that the QTL position embedded in the haplotype is IBD, conditional on the marker information. They were able to compute these probabilities using a genomic drop simulation. A later paper describes a deterministic method, based on coalescent theory, to arrive at these probabilities (Meuwissen and Goddard. 2001. Genetics Selection Evolution 33: 605-634). Both methods to compute IBD probabilities assume that the number of generations to a common ancestor and the effective population size of the founder generation are known. They also assume one single population that has grown in isolation since it was founded. Present day livestock populations are most likely the result of more complex evolutionary processes, such as bottlenecks, admixture and inbreeding. Coalescent theory (Kingman, Stochastic Processes and Their Applications 13: 235-248, 1982) could be further utilized to better understand the evolutionary dynamics of studied populations.

In general, traditional linkage studies provide an opportunity to trace inheritance within families. Differences between the phenotypic means of offspring groups inheriting alternative marker alleles indicate which marker alleles are linked to QTL alleles. Many statistical techniques based on either linear regression or maximum likelihood are used to analyze the data. Recombination between marker alleles and QTL alleles can be taken account of by factoring recombination into the likelihood or regression coefficients.

A family based design in a genetic association study is likely to be more complex. The families are likely to have complex genealogies for which traditional linkage calculations are not computationally feasible. One option is to ignore family structure and operate on the same premise as population based designs. The potential problem with this strategy is similar to the problem of confounding due to stratification caused by breed type. Shared background genes and shared environmental effects may cause individuals within families to display similar phenotypic variation. Any SNPs that are in high frequency in that family are potentially associated with the trait.

Human geneticists devised the transmission disequilibrium test (TDT) to avoid spurious population associations caused by ethnic stratification of a sample of people affected by a disease (Terwilliger and Ott. 1992. Human Heredity 42: 337-346; Spielman et. al. 1993. American Journal of Human Genetics 52, 506-516; Rabinowitz. 1997. Human Heredity 47: 342-350). In families, certain phase combinations of marker and QTL alleles exist on parental chromosomes. Because the loci are linked, these combinations will be preferentially transmitted from parent to child. In contrast, marker alleles associated with the trait due to stratification, but unlinked to the QTL, are not preferentially transmitted to children.

The same principle can be used in association studies in livestock where family based designs are used. That is, associations between SNPs and a quantitative trait should be conditioned on parental genotypes. Separate transmission disequilibrium tests could be performed at each SNP. This approach will encounter multiple testing problems and likely result in overestimation of allelic effects. The TDT is also restricted to within family information, which will affect the power of the test. A more powerful approach would be to analyze chromosomal segments and condition the variance covariance matrix of haplotype effects on parental information. Recently Meuwissen et al. (2002. Genetics 161: 373-379) outlined such a method. Essentially if two haplotypes occur in animals with a known common ancestor, then the calculation of IBD probability is modified to account for this. However this method is restricted to analyzing one segment at a time, as in interval mapping.

A further refinement to this procedure would be to analyze all segments simultaneously. This would involve computing many hundreds of variance-covariance matrices, each of which can be of considerable order. Blott et al. (2003. Genetics 163: 253-266) propose reducing the number of observed haplotypes into clusters using distance matrix methods such as UPGMA. Such an approach would aid in reducing the computational burden when analyzing all segments simultaneously.

Gianola et al. (2003 Genetics 163: 347-365) suggest extending the modeling of phenotypic-marker associations by including chromosomal effects, spatial covariance of marked effects within chromosomes and family heterogeneity. The techniques suggested have merit and should be examined.

Prior to phenotypic analysis the following are completed: a) the resource population is scrutinized for population stratification using appropriate software (e.g. Pritchard et al. Genetics 155: 945-959); b) Hardy Weinberg disequilibrium (HWD) are measured for each SNP; c) two-locus linkage disequilibrium (LD) metrics (D prime and R2) are computed for all pair-wise SNP combinations and LD correlated with physical distance; and d) two-locus sample probabilities are computed under various evolutionary models (Hudson 2001 Genetics 159: 1805-1817; McVean et al. 2002 Genetics 160: 1231-1241) in order to determine whether the observed level of linkage disequilibrium is unusually large or small, and to estimate recombination rate variation across the genome.

Phenotypic analysis can involve both classical and Bayesian approaches. The classical approach consists of performing least squares regressions on all SNP separately and on small sets of SNP. A hierarchy of models are then tested. The standard model is one that does not assume heterogeneity of variance due to chromosomal structure. The hierarchy begins by partitioning the variance of marker effects between chromosomes; then by introducing a covariance structure which accounts for the possibility that adjacent within-chromosome effects are more strongly correlated that those further apart. In the case of family-based designs a full relationship matrix under additive inheritance will be introduced in order to account for polygenic effects. In addition the model can be extended to include chromosome and within-chromosome effects that are family specific. Permutation tests will be used to derive significance thresholds.

The Bayesian approach can be completed using a Markov Chain Monte Carlo approach. The same hierarchy of models that were tested in the classical setting can be used, except that all SNP can be now included in the one analysis. A suitable variable selection procedure can be included in the Bayesian regression set up in order to identify models with the highest posterior probability. The model which fits the effects of haplotype could also be used and once the best model has been identified molecular genetic values (MGVs) can be computed for individuals. MGVs combine the individual marked effects into one total molecular score. Each MGV includes an associated accuracy.

Current methods for selection of grandparents and parents of commercial birds or avian species are based on the birds own performance for traits and the performance of their progeny and other relatives. The information is compiled to estimate the genetic merit of an individual bird or avian species. In order to get an accurate prediction of the genetic merit, the animal's phenotype and progeny must be measured. These methods are costly and time consuming. In one embodiment, MGV's birds or avian species could be selected at birth based on their SNP marker genotype. Only those birds with the best genotype would be selected to be parents of the next generation. Subsequently, birds with the best MGV's for specific markets and customers are identified and utilized to create market specific animals. Parents are selected to optimize hybrid vigor in the commercial birds or avian species. Progeny resulting from mating of selected parents would contain the optimum combination of traits, thus creating an enduring genetic pattern and line of animals with specific traits. These lines are monitored for purity using the original SNP markers and identified from the entire population of non-beef livestock and protected from genetic theft.

In another embodiment, commercial birds or avian species are cloned based on their genetic potential for a specific trait or series of traits. Birds or avian species are tracked for historical and epidemiological reasons, and the location of an animal from embryo to birth through its growth period, to harvest and finally the retail product after it has reached the consumer could be monitored.

The results of the present whole-genome association study can be used to select parents of commercial birds, make decisions concerning the animals to mate to produce commercial birds and produce branded products for growers or processors. These tools could be used to assess health condition for resistance to disease or infection, susceptibility to infection with and shedding of pathogens such as E. coli, Salmonella, Listeria, and other organisms potentially pathogenic to humans, or regulation of immune status and response to antigens.

Provided herein are methods for inferring a trait of a non-beef livestock subject from a nucleic acid sample obtained from the subject. Although many of the descriptions recite nucleic acids isolated from a chicken subject, these descriptions are made for convenience and to avoid redundancies. Therefore, the method is not to be construed as limited to inferring traits of chicken livestock but rather to be read on the identification of certain traits in any non-beef livestock subject according to the present methods.

Although the invention has been described with reference to the above example, it will be understood that modifications and variations are encompassed within the spirit and scope of the invention. Accordingly, the invention is limited only by the following claims.

1-208. (canceled)

209. A method for inferring a trait of a non-beef subject from a nucleic acid sample of the non-beef subject, the method comprising identifying in the nucleic acid sample at least one nucleotide occurrence of at least one single nucleotide polymorphism (SNP) corresponding to the nucleotide at position 600 of any one of SEQ ID NOs:1-96,631, or the complement thereof, thereby identifying a trait of the non-beef subject.

210. The method of claim 209, wherein the nucleotide occurrence of at least 2 SNPs is determined.

211. The method of claim 210, wherein the at least 2 SNPs comprise a haploytpe, wherein the method identifies a haplotype allele that is associated with the trait.

212. The method of claim 211, comprising identifying a diploid pair of haplotype alleles.

213. The method of claim 209, wherein the non-beef livestock subject is an avian species.

214. The method of claim 213, wherein the avian species is a chicken or a turkey.

215. The method of claim 213, wherein the trait is egg production, feed efficiency, livability, meat yield, white meat yield, dark meat yield, disease resistance, disease susceptibility, optimal diet time to maturity, time to a target weight, weight at a target timepoint, average daily weight gain, meat quality, muscle content, fat content, feed intake, protein content, bone content, maintenance energy requirement, mature size, amino acid profile, fatty acid profile, stress susceptibility and response, digestive capacity, production of calpain, calpastatin activity and myostatin activity, pattern of fat deposition, fertility, ovulation rate, or conception rate.

216. The method of claim 213, wherein the trait is resistance to Salmonella infection, ascites, and listeria infection.

217. The method of claim 213, wherein the avian species is a species that produces eggs for mammalian consumption.

218. The method of claim 214, wherein the avian species is a chicken.

219. The method of claim 218, wherein the trait is a characteristic of an egg of the avian species or a characteristic of a product of the egg.

220. The method of claim 219, wherein the egg characteristic is quality, size, shelf life, freshness, cholesterol content, color, biotin content, calcium content, shell quality, yolk color, lecithin content, number of yolks, yolk content, white content, vitamin content, vitamin D content, nutrient density, protein content, albumen content, protein quality, avidin content, fat content, saturated fat content, unsaturated fat content, interior egg quality, number of blood spots, air cell size, grade, a bloom characteristic, chalaza prevalence or appearance, ease of peeling, likelihood of being a restricted egg, Salmonella content.

221. The method of claim 209, wherein the sample is isolated from a tissue or a bodily fluid.

222. The method of claim 209, wherein the target nucleic acid molecule is a DNA molecule.

223. The method of claim 222, wherein the DNA molecule is genomic DNA.

224. The method of claim 222, wherein the DNA molecule is double-stranded DNA.

225. The method of claim 222, wherein the DNA molecule is single-stranded DNA.

226. The method of claim 209, wherein the nucleic acid molecule is an RNA molecule.

227. A method for identifying the parentage of a non-beef test subject, the method comprising:
a) obtaining a nucleic acid sample from the test subject by a method comprising identifying in the nucleic acid sample at least one single nucleotide polymorphism (SNP) corresponding to the nucleotide at position 600 of any one of SEQ ID NOs:1-96,631, or the complement thereof;
b) optionally repeating a) for additional subjects;
c) determining the alleles corresponding to each SNP identified; and
d) comparing the alleles to putative parents of the test subject
wherein parents not possessing at least one allele in common with the test subject is excluded.

228. A method for determining the identity of a non-beef test subject, the method comprising:
a) obtaining a nucleic acid sample from the test subject by a method comprising identifying in the nucleic acid sample at least one single nucleotide polymorphism (SNP) corresponding to the nucleotide at position 600 of any one of SEQ ID NOs:1-96,631, or the complement thereof;
b) optionally repeating a) for additional subjects;
c) determining the two alleles corresponding to each SNP identified; and
d) comparing the alleles to the alleles identified in a known sample previously obtained from the test subject.

229. A method to infer breed or line of a non-beef test subject from a nucleic acid sample obtained from the subject, comprising identifying in the nucleic acid sample, at least one nucleotide occurrence of at least one single nucleotide polymorphism (SNP) corresponding to the nucleotide at position 600 of any one of SEQ ID NOS:1-96,631, wherein the SNP is associated with a breed, thereby inferring the breed of the non-beef subject.

230. A method of generating a genome discovery map comprising:
a) selecting a plurality of single nucleotide polymorphism (SNP) markers selected from at least two of the SNP markers at position 600 of any of SEQ ID NOs:1-96,631, wherein each marker in the series will be separated by approximately 150,000 bp; and
b) generating the genome discovery map based upon the selected markers.

231. The method of claim 230, wherein the genome discovery map is a whole genome discovery map.

232. The method of claim 230, wherein the plurality of single nucleotide polymorphism (SNP) markers includes about 10 markers.

233. The method of claim 230, wherein the plurality of single nucleotide polymorphism (SNP) markers includes about 100 markers.

234. The method of claim 230, wherein the plurality of single nucleotide polymorphism (SNP) markers includes about 1000 markers.

235. The method of claim 230, wherein the plurality of single nucleotide polymorphism (SNP) markers includes about 8000 markers.

236. The method of claim 230, wherein the plurality of single nucleotide polymorphism (SNP) markers includes about 10000 markers.

237. The method of claim 228, wherein the non-beef livestock subject is a avian species.

238. The method of claim 237, wherein the avian species is a chicken or a turkey.

239. The method of claim 238, wherein the avian species is a chicken.

240. The method of claim 230, wherein the plurality of single nucleotide polymorphism (SNP) markers, or the number of markers indicated by the amount of linkage disequilibrium in each non-beef species, are further selected based upon dispersion across the entire genome.

241. (canceled)

242. (canceled)

243. A database comprising a plurality of single nucleotide polymorphisms (SNP) selected from at least two of the SNP markers at position 600 of any of SEQ ID NOs:1-96,631, or complement thereof.

244. A database comprising the alleles frequencies generated by analyzing the database of claim 243.

245. An isolated single nucleotide polymorphism (SNP) corresponding to a nucleotide at position 600 of any one of SEQ ID NOs:1-96,631, or the complement thereof.

246-249. (canceled)

250. A panel comprising at least one single nucleotide polymorphism (SNP) corresponding to a nucleotide at position 600 of any one of SEQ ID NOs:1-96,631, or the complement thereof.

251. (canceled)
